(19) United States
(12) Patent Application Publication
(10) Pub. No.: US 2016/0172066 A1
(43) Pub. Date: Jun. 16, 2016

(54) ENERGY DEGRADER

(71) Applicant: ION BEAM APPLICATIONS S.A., Louvain-la-Neuve (BE)

(72) Inventor: Yves CLAEREBOUDT, Nil-Saint-Vincent (BE)

(21) Appl. No.: 14/971,992

(22) Filed: Dec. 16, 2015

(30) Foreign Application Priority Data
Dec. 16, 2014 (EP) 14198364.3

(51) Int. Cl. G21K 1/00

(52) U.S. Cl. CPC G21K 1/00; A61N 2005/1095

(57) ABSTRACT

Disclosed embodiments include an energy degrader for attenuating the energy of a charged particle beam and comprising two energy attenuation members having different masses. The degrader further comprises a drive unit configured to move simultaneously the two energy attenuation members at respectively a first and a second speed across the particle beam during a first movement and to move the lightest of the two energy attenuation members at a third speed across the particle beam during a second movement, the third speed being higher than the first speed. More accurate and faster variation of the energy of the charged particle beam can hence be achieved.

### CROSS-REFERENCE TO RELATED APPLICATION

This U.S. patent application claims priority under 35 U.S.C. §119 to: European Patent Application No. EP14198364.3, filed Dec. 16, 2014. The aforementioned application is incorporated herein by reference in its entirety.

### TECHNICAL FIELD

This disclosure relates to the field of charged particle accelerators, such as proton or carbon ion accelerators for example, and more particularly to an energy degrader for attenuating the energy of a charged particle beam produced by such a particle accelerator.

### BACKGROUND

Certain applications involving the use of beams of charged particles may require the energy of these particles to be varied. This is for example the case in particle therapy applications, where the energy of the charged particles determines the depth of penetration of these particles into a body to be treated by such therapy.

Some charged particle accelerators, such as synchrotrons for instance, are adapted to vary the energy of the particle beam which they produce, but it may nevertheless be desirable to further vary the energy after the particles have been extracted from the synchrotron. Other particle accelerators, such as cyclotrons for instance, are not themselves adapted to vary the energy of the particle beam which they produce, and therefore may require an additional device to vary this energy.

Devices for varying the energy of a particle beam extracted from a particle accelerator are often called energy degraders. An energy degrader comprises therefore one or more blocks of matter which are placed across the path of the particle beam after its extraction from the particle accelerator. According to a well-known principle, any particle passing through such a block of matter undergoes a decrease in its energy by an amount which is, for particles of a given type, a function of the intrinsic characteristics of the material passed through and of its thickness.

Known energy degraders may include a single block of matter which has the shape of a helicoidal staircase. The particle beam enters the degrader perpendicularly to a step of the staircase and exits the degrader at an opposite side, which attenuates the energy of the beam according to the thickness of the degrader at said step. After having rotated the staircase by a given angle around its axis, the beam will enter the degrader perpendicularly to another step, which will attenuate the energy of the beam by a different amount according to the thickness of the degrader at said other step. The energy attenuation can thus varied by changing the angular position of the degrader with respect to the particle beam.

Existing energy degraders may also include two wedge-shaped blocks of matter which are transversally movable into the beam path. The energy attenuation is varied by changing the transversal position of the blocks with respect to the particle beam.

### SUMMARY

A drawback of known energy degraders is that they are heavy and that it is therefore difficult to move them quickly and/or with high accuracy with respect to the particle beam. Some recent applications require however to be able to change the energy of the particle beam very quickly, such as in a few tens of milliseconds for instance, and/or with high positional accuracy.

This is for example the case with particle therapy systems, where a target, such as a tumour for example, is to be irradiated layer by layer with the particle beam, these layers being at different depths into the body of the patient. In such cases, it is desirable to be able to change the energy of the particle beam very quickly and/or very accurately when the system passes from one layer to another layer.

Disclose embodiments may provide improvements over existing energy degraders. For example, disclose embodiments may provide an energy degrader which is adapted to vary the energy of a particle beam more quickly and/or with higher accuracy than the known degraders.

A typical beam energy upstream (i.e. at an input) of an energy degrader according to disclose embodiments is in the MeV range, such as in the range of 150 MeV to 300 MeV for example, and a typical desired beam energy downstream (i.e. at an output) of the energy degrader according to disclosed embodiments is also in the MeV range, such as in the range of 50 MeV to 230 MeV for an input energy of 230 MeV for example.

According this disclosure, there is provided an energy degrader for attenuating the energy of a charged particle beam, such as a beam of protons or of carbon ions for example, said energy degrader comprising:
a first energy attenuation member (A) adapted to attenuate the energy of charged particles crossing said first attenuation member,
a second energy attenuation member (B) adapted to attenuate the energy of charged particles crossing said second attenuation member, and
a drive unit operably connected to the first and to the second energy attenuation members and configured for moving the first and/or the second energy attenuation members across the charged particle beam,
   wherein:
the mass (m_{A}) of the first energy attenuation member (A) is smaller than the mass (m_{B}) of the second energy attenuation member (B),
the drive unit is configured for moving simultaneously the first energy attenuation member (A) at a first speed (VA1) and the second energy attenuation member (B) at a second speed (VB1) across the charged particle beam during a first movement,
the drive unit is configured for moving the first energy attenuation member (A) at a third speed (VA2) across the charged particle beam during a second movement, and
the average third speed (VA2) over the second movement is larger than the average second speed (VB1) over the first movement.

By moving the lighter energy attenuation member (A) at the higher speed (VA2) across the charged particle beam during the second movement, one can indeed vary the total energy attenuation quickly and with higher accuracy than with the conventional energy degraders. In a particle therapy system for instance, this second movement and the accompanying variation of the energy of the particle beam may for instance be performed during a change of layer of the target to be irradiated.

Moreover, by moving simultaneously the lighter energy attenuation member (A) at the first speed (VA1) and the heavier energy attenuation member (B) at the lower speed (VB1) across the charged particle beam during the first movement, one may reposition the lighter energy attenuation member (A) with respect to the particle beam so that it is ready for the next second movement, yet without having to move the heavier mass (m_{B}) quickly. The shapes of the first and the second energy attenuation members are moreover preferably designed so that a total energy attenuation of the particle beam remains constant during the first movement. In a particle therapy system for instance, this first movement may for instance be performed in the course of the irradiation of a given layer of the target.

Preferably, the mass (m_{A}) of the first energy attenuation member is smaller than 0.5 times the mass (m_{B}) of the second energy attenuation member.

More preferably, the mass (m_{A}) of the first energy attenuation member is smaller than 0.1 times the mass (m_{B}) of the second energy attenuation member Even more preferably, the mass (m_{A}) of the first energy attenuation member is smaller than 0.02 times the mass (m_{B}) of the second energy attenuation member.

Preferably, the first energy attenuation member is made of the same material as the second energy attenuation member.

Preferably, the average third speed (VA2) over the second movement is larger than two times the average second speed (VB1) over the first movement. More preferably, the average third speed (VA2) over the second movement is larger than five times the average second speed (VB1) over the first movement. Even more preferably, the average third speed (VA2) over the second movement is larger than ten times the average second speed (VB1) over the first movement.

According to the disclosure, there is preferably provided an energy degrader for attenuating the energy of a charged particle beam, said energy degrader comprising:
a first energy attenuation member (A) adapted to attenuate the energy of charged particles crossing said first attenuation member and having the shape of a wedge presenting a first beam entry face (A1) and a an opposed first beam exit face (A2),
a second energy attenuation member (B) adapted to attenuate the energy of charged particles crossing said second attenuation member and having the shape of a wedge presenting a second beam entry face (B1) and an opposed second beam exit face (B2),
said first and second beam entry faces as well as said first and second beam exit faces being flat faces,
a drive unit operably connected to the first and to the second energy attenuation members and configured for moving the first and/or the second energy attenuation members across the charged particle beam,
   wherein:
the mass (m_{A}) of the first energy attenuation member (A) is smaller than the mass (m_{B}) of the second energy attenuation member (B),
the drive unit is configured for moving simultaneously the first energy attenuation member (A) at a first speed (VA1) and the second energy attenuation member (B) at a second speed (VB1) across the charged particle beam during a first translational movement,
the drive unit is configured for moving the first energy attenuation member (A) at a third speed (VA2) across the charged particle beam during a second translational movement, and
the average third speed (VA2) over the second movement is larger than the average second speed (VB1) over the first movement.

Such a preferred configuration allows indeed for much more flexibility in the variation of the attenuation of the energy of the particle beam, compared to cases where the energy attenuation members have other shapes and/or compared with cases where the movements of the energy attenuation members are not translational movements.

In such a preferred configuration, the first beam entry face (A1) is preferably parallel to the second beam exit face (B2), the first beam exit face (A2) is preferably parallel to the second beam entry face (B1), and the drive unit is preferably configured in such a way that, during the first movement, the instantaneous first speed (VA1) is equal to the instantaneous second speed (VB1). This allows for the particle beam to enter and exit the energy degrader perpendicularly to the first entry face and to the last exit face, thereby reducing unwanted distortions on the particle beam.

According to the disclosure, there is also provided a particle therapy system comprising a particle accelerator configured for producing a charged particle beam, and comprising an energy degrader according to the disclosure for attenuating the energy of the charged particle beam output by the particle accelerator. The particle accelerator is preferably a fixed-energy accelerator, more preferably a cyclotron, for example a synchrocyclotron.

### SHORT DESCRIPTION OF THE DRAWINGS

These and further aspects of the disclosure will be explained in greater detail by way of example and with reference to the accompanying drawings in which:

FIG. 1 schematically shows an energy degrader according to disclosed embodiments;

FIG. 2 schematically shows an energy degrader according to disclosed embodiments, with an exemplary drive unit;

FIG. 3 schematically shows an energy degrader according to disclosed embodiments, with a preferred drive unit;

FIG. 4 schematically shows a preferred energy degrader according to disclosed embodiments;

FIGS. 5a, 5b, 5c schematically show a cross section of the energy degrader of FIG. 4 at various stages of a movement of its components;

FIG. 6 schematically shows a more preferred energy degrader according to disclosed embodiments;

FIG. 7 schematically shows an even more preferred energy degrader according to disclosed embodiments;

FIGS. 8a, 8b, 8c, 8d schematically show cross sections of the energy degrader of FIG. 7 and according to different arrangements of its components and of their movements;

FIG. 9 schematically shows a variant of an energy degrader according to disclosed embodiments;

FIG. 10 schematically shows another variant of an energy degrader according to disclosed embodiments;

FIG. 11 schematically shows a part of a particle therapy system comprising a particle accelerator and an energy degrader according to disclosed embodiments.

The drawings of the figures are neither drawn to scale nor proportioned. Generally, similar or identical components are denoted by the same reference numerals in the figures.

### DETAILED DESCRIPTION

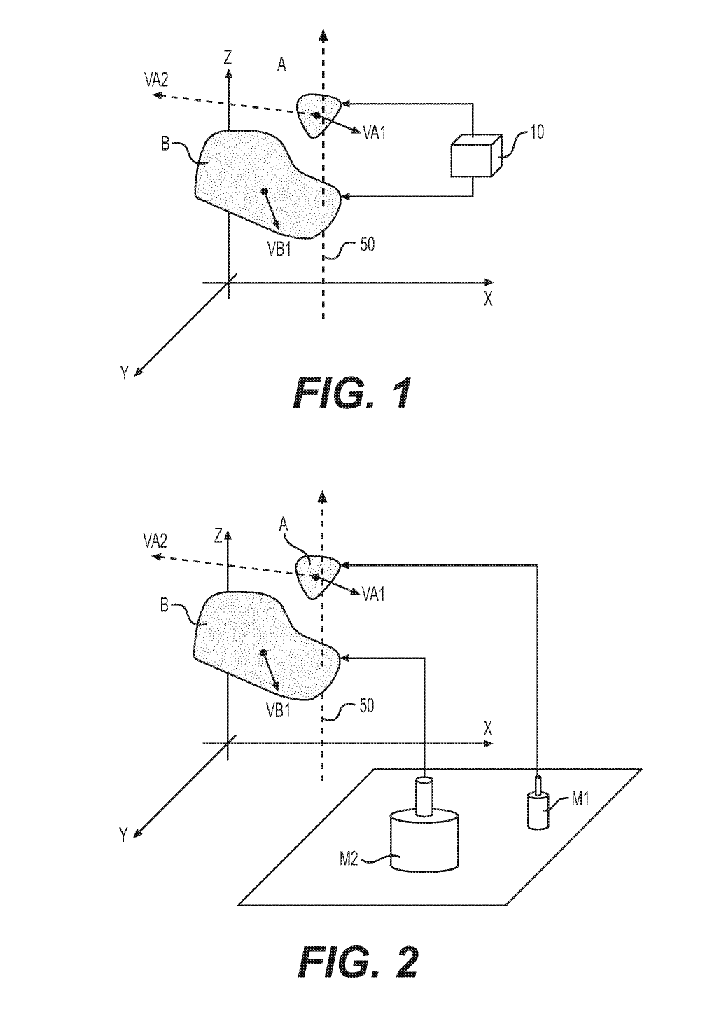

FIG. 1 schematically shows a 3D view of an energy degrader according to the disclosure. The energy degrader comprises a first energy attenuation member (A) adapted to attenuate the energy of charged particles crossing said first attenuation member and a second energy attenuation member (B) adapted to attenuate the energy of charged particles crossing said second attenuation member. As is well known in the art, these attenuation members are for example blocks of solid matter such as blocks of Beryllium or of Carbon graphite for example. Preferably, the first energy attenuation member (A) is made of the same material as the second energy attenuation member (B). Specific to the disclosure is that the mass (m_{A}) of the first energy attenuation member (A) is smaller than the mass (m_{B}) of the second energy attenuation member (B). Preferably, m_{A} is smaller than 0.5 times the m_{B}. More preferably, m_{A} is smaller than 0.1 times the m_{B}. Even more preferably, m_{A} is smaller than 0.02 times the m_{B}. Exemplary masses will be given hereafter.

A charged particle beam (50) crossing both the first and the second energy attenuation members is shown on FIG. 1. In operation, the energy degrader will thus attenuate the energy of the particle beam (50) following to its crossing of the both the first and the second energy attenuation members, as shown on FIG. 1. A total attenuation of the energy of the particle beam (50) may be estimated as the sum of the energy attenuations provided by the first and the second energy attenuation members along the path of the particle beam (50). The energy degrader further comprises a drive unit (10) which is operably connected to the first and to the second energy attenuation member for moving the first and/or the second energy attenuation members across the charged particle beam (50). Such drive units are also well known in the art. The drive unit (10) is configured for moving simultaneously the first energy attenuation member (A) at a first speed (VA1) and the second energy attenuation member (B) at a second speed (VB1) across the charged particle beam (50) during a first movement. Depending on the shapes of the energy attenuation members and on their respective speeds, the energy attenuation will vary or not vary in the course of this first movement. Preferred examples of speeds and shapes resulting in the total energy attenuation remaining constant during this first movement will be given hereafter.

The drive unit (10) is further configured for moving the first energy attenuation member (A) at a third speed (VA2) across the charged particle beam (50) during a second movement. In the course of this second movement, the second energy attenuation member (B) may or may not move. However, the shapes and the positions of the first and second energy attenuation members shall preferably be chosen in such a way that, when in operation, the particle beam (50) crosses both energy attenuation members during the first movement as well as during the second movement.

Specific to the disclosure is that the average third speed (VA2) over the second movement is larger than the average second speed (VB1) over the first movement. Preferably, the average third speed (VA2) over the second movement is larger than two times the average second speed (VB1) over the first movement. More preferably, the average third speed (VA2) over the second movement is larger than five times the average second speed (VB1) over the first movement. Even more preferably, the average third speed (VA2) over the second movement is larger than ten times the average second speed (VB1) over the first movement. For a particle therapy application, it is for example advantageous and found to be feasible that VB1 is in the range of 0.02 m/s to 0.2 m/s (for example 10 cm/5 s to 10 cm/500 ms), preferably in the range of 0.05 m/s to 0.1 m/s (for example 10 cm/2 s to 10 cm/1 s), and that VA2 is in the range of 0.2 m/s to 100 m/s (for example 10 cm/500 ms to 10 cm/1 ms), preferably in the range of 1 m/s to 2 m/s (for example 10 cm/100 ms to 10 cm/50 ms).

In any and all of these cases, the drive unit (10) is preferably configured in such a way that, at any instant in the course of the first movement, the instantaneous first speed (VA1) is equal to the instantaneous second speed (VB1) (both speeds being considered as vector quantities in this case). This allows indeed for the total energy attenuation to depend solely or substantially on the shapes of the first and second energy attenuation members, which simplifies the design of the system.

FIG. 2 schematically shows an energy degrader according to the disclosure, with an exemplary drive unit (10). In this example, a first motor (M1) is operably connected to move the first energy attenuation member (A) at the first speed (VA1) during the first movement and at the third speed (VA2) during the second movement, with respect to a stationary part—such as a chassis for example—or to the particle beam (50). A second motor (M2) is operably connected to move the second energy attenuation member (B) at the second speed (VB1) during the first movement, with respect to the stationary part or to the particle beam (50).

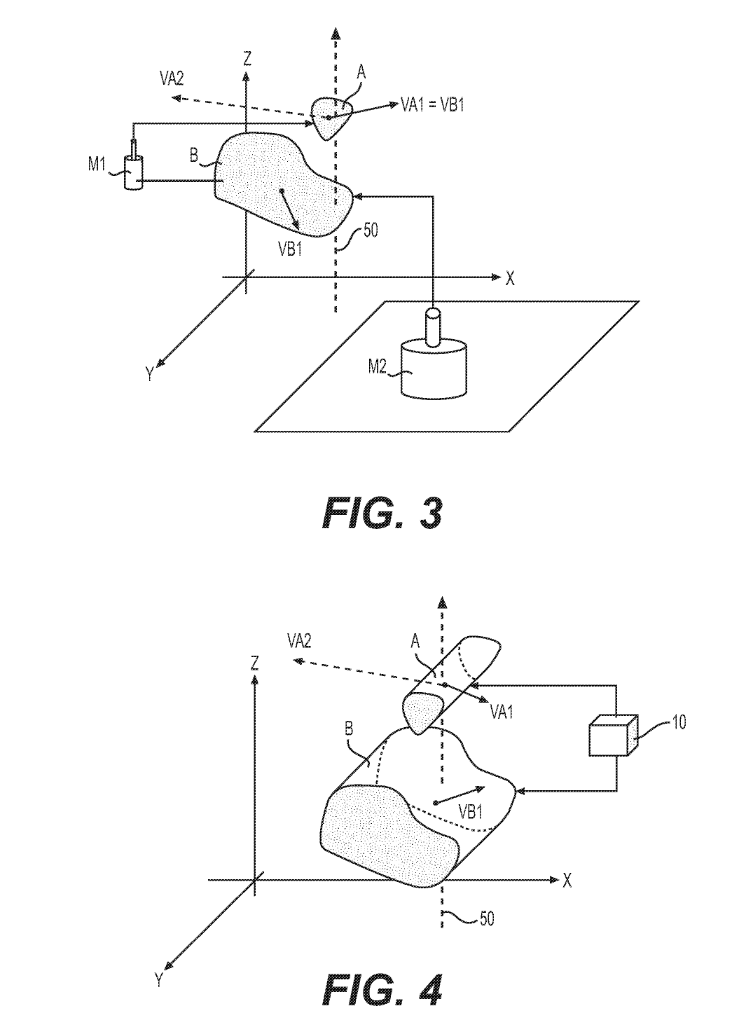

FIG. 3 schematically shows a preferred energy degrader according to the disclosure, with a preferred drive unit (10). In this example, a second motor (M2) is operably connected to move the second energy attenuation member (B) at the second speed (VB1) during the first movement, with respect to a stationary part—such as a chassis for example—or to the particle beam (50). A first motor (M1), whose stator is rigidly connected to the second energy attenuation member (B), is operably connected to move the first energy attenuation member (A) at the first speed (VA1) during the first movement and at the third speed (VA2) during the second movement, with respect to the second attenuation member. Hence, by operating the second motor (M2) while not operating or while blocking the first motor (M1), both the first and the second energy attenuation members will move simultaneously and at the same speed with respect to the chassis or to the particle beam (50) during the first movement, and, by operating the first motor (M1), the first energy attenuation member (A) will move with respect to the chassis or to the particle beam (50) during the second movement.

In the examples given hereafter and wherein the energy attenuation members are to be moved into translation, and in case the motor is a rotating type motor such as an electric motor for instance, one will of course make use of an intermediary transmission (not shown) in order to transform the rotational movement of the motor(s) into a translational movement applied to the energy attenuation members (A, B). In the examples given hereafter and wherein the energy attenuation members are to be moved into rotation, and in case the motor is a rotating type motor, one may also use an intermediary transmission (not shown) in order to adapt the speed and/or the torque applied to the energy attenuation members (A, B).

FIG. 4 schematically shows a 3D view of a preferred energy degrader according to the disclosure in an XYZ referential. As can be seen on this figure, the first and the second energy attenuation members each have a cylindrical shape (a shape obtained by moving a straight line parallel to itself, in this example parallel to the Y axis). In this case, the drive unit (10) is preferably configured to move the first and the second energy attenuation members in translation in a plane parallel to the XZ plane (vectors VA1 and VA2 are parallel to XZ).

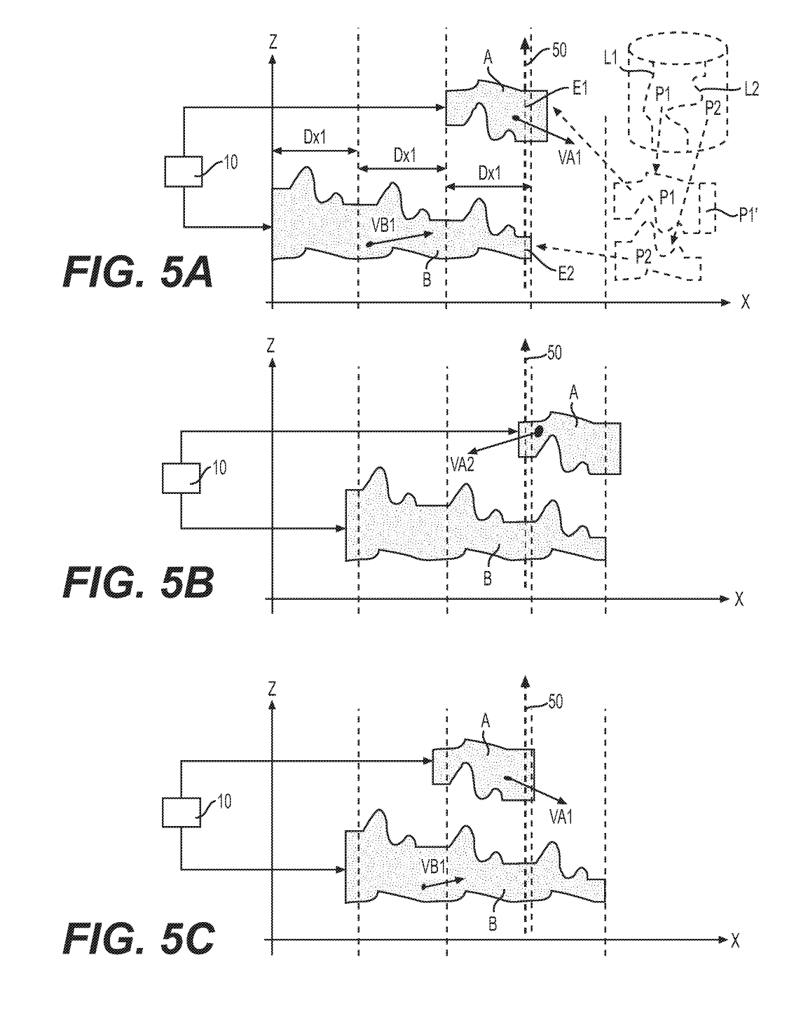

FIGS. 5a, 5b, 5c schematically show cross sections of an exemplary and preferred energy degrader according to FIG. 4, at various stages of a movement of the first and the second energy attenuation members, the cross sections being taken according to a plane parallel to the XZ plane. In these figures, an imaginary particle beam (50) is show whose path is parallel to the Z axis. Such preferred energy degrader is particularly useful for particle therapy, as will become clearer hereafter.

The cross sections are special here. They are obtained by dividing the surface of an imaginary cylinder along two freely chosen lines (L1, L2), yielding thus two imaginary parts (P1, P2), and by deploying these two parts so that they become flat, as shown at the right side of FIG. 5a in dashed lines. A flat rectangular appendix (P1′) is moreover added to the right side of the first part (P1). P1 and P1′ together represent the cross section of the first energy attenuation member (A).

The second part (P2) represents the cross section of the rightmost part of the second energy attenuation member (B), namely whose width equals to Dx1. The two other parts of the second energy attenuation member (B) which are at the left of this rightmost part each have a width which equals to Dx1. They are obtained by increasing the height of the second part (P2) without changing the top and bottom profiles of the second part and by respectively aligning the corresponding top and bottom profiles.

The way the drive unit (10) moves the first and the second energy attenuation members will now be described in more detail.

FIG. 5a shows the positions of the first and the second energy attenuation members with respect to the particle beam (50) in an initial condition for instance. The left side of the rightmost part of the second energy attenuation member (B) is vertically aligned with the left side of the first energy attenuation member (A). The drive unit (10) is configured for moving simultaneously the first energy attenuation member (A) at a first speed (VA1) and the second energy attenuation member (B) at a second speed (VB1) across the charged particle beam (50) during a first movement. Specific here is that, during said first movement, the horizontal (X) component of the instantaneous first speed (VA1) is equal to the horizontal (X) component of the instantaneous second speed (VB1). This can for example be obtained easily by using a drive unit (10) as shown in FIG. 3 and by blocking the first motor (M1) at standstill during the first movement, so that the first energy attenuation member (A) does not move with respect to the second energy attenuation member (B).

Thanks to the special cross sections of the first and the second energy attenuation members as described hereinabove (see parts P1+P1′ and P2) and thanks to their equal instantaneous speeds in the X direction, it will be easily understood that a total distance traveled by the particles of the particle beam (50) through the first and the second energy attenuation members—and therefore a total energy attenuation of these particles by the energy degrader—remains constant during the first movement.

FIG. 5b shows the positions of the first and the second energy attenuation members with respect to the particle beam (50) at an end of the first movement, namely when the first and the second energy attenuation members have moved over a horizontal (X) distance which is preferably a little smaller than Dx1 so that the particle beam (50) still crosses both the first and the second energy attenuation members at the end of the first movement.

The drive unit (10) is further configured for then moving the first energy attenuation member (A) at a third speed (VA2) across the charged particle beam (50) during a second movement, the average third speed (VA2) over the second movement being larger than the average second speed (VB1) over the first movement. In this example, the second movement is opposite in direction to the first movement.

FIG. 5c shows the positions of the first and the second energy attenuation members with respect to the particle beam (50) at an end of the second movement, namely when the first energy attenuation member (A) has moved to the left over a horizontal (X) distance of Dx1, so that the first energy attenuation member (A) is again positioned in front of the mating part of the second energy attenuation member (B). At this point in time, a total distance traveled by the particle beam (50) through the first and the second energy attenuation members—and therefore a total energy attenuation of the particle beam (50)—will be larger than during the first movement (FIGS. 5a and 5b).

From this point in time, one may repeat a sequence of the first and second movements described in relation to FIGS. 5a and 5b, as many times as necessary, provided one does not exceed the total width (X) of the second energy attenuation member (B) of course.

This configuration is particularly useful for particle therapy systems, where a target (200), such as a tumour for example, is to be irradiated layer by layer with a particle beam (50), these layers being at different depths into the body of the patient. In such cases, it is desirable to be able to change the energy of the particle beam (50) very quickly and/or very accurately when the system passes from one layer to another layer. The configuration of FIGS. 5a, 5b and 5c permits to achieve this in the following way. First, the energy degrader is positioned with respect to the particle beam (50) as shown in FIG. 5a. The particle beam (50) is then turned on to irradiate a first layer of the tumour (e.g. the deepest layer). While irradiating said layer, the drive unit (10) performs the first movement of the first and the second energy attenuation members at a relatively low speed (VA1x=VA2x). As explained, the total energy attenuation—and therefore the energy of the beam at the exit of the energy degrader—remains constant during this first movement. At the end of the first movement (FIG. 5b), the beam is preferably turned off. While the beam is off, the drive unit (10) performs the second movement of the first energy attenuation member (A) at a relatively high speed (VA2). At the end of the second movement (FIG. 5c), the beam is turned on again. At this point in time, the total energy attenuation—and therefore the energy of the beam at the exit of the energy degrader—is lower than in the course of the first movement; another layer of the tumour, less deep than the first layer, can then start to be irradiated. Since the mass (m_{A}) of the first energy attenuation member (A) is smaller than the mass (m_{B}) of the second energy attenuation member (B), the second movement can be made very quickly and with high accuracy. This reduces the time needed for changing the beam energy between two layers, which reduces the treatment time.

It will be obvious that one may also proceed the other way around, i.e. by initially positioning the first energy attenuation member (A) right above the matching portion of the thickest part of the second energy attenuation member (B) (left side in FIG. 5a) and to move the first and second energy attenuation member to the left during the first movement, and then to move the first energy attenuation member (A) to the right during the second movement.

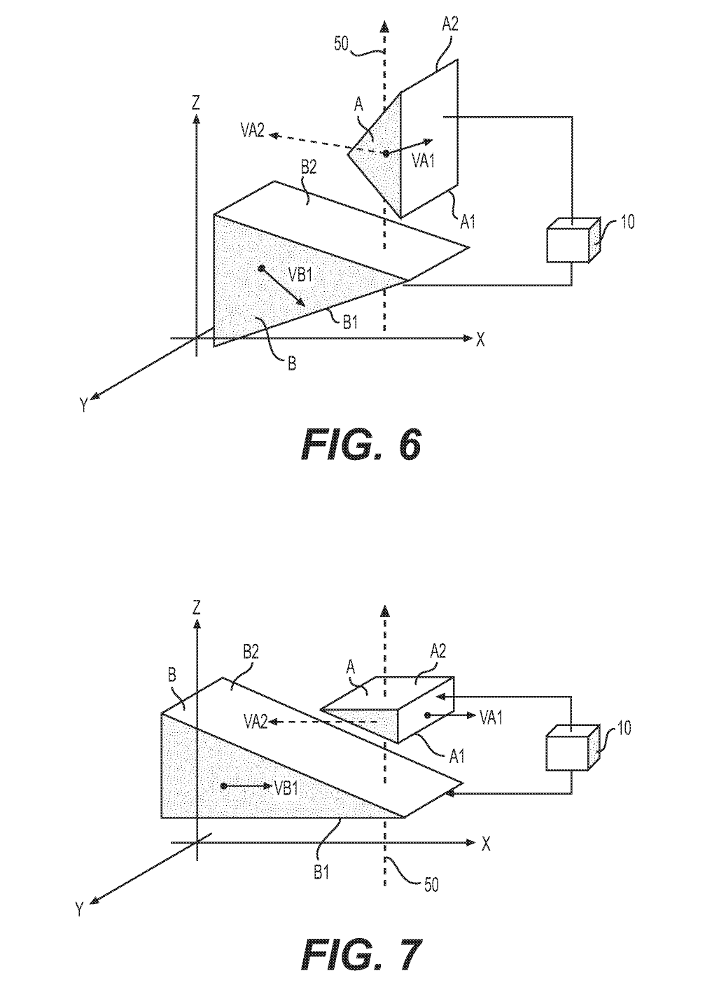

FIG. 6 schematically shows a 3D view a more preferred energy degrader according to the disclosure. It is identical to the ones described hereinabove except that the first energy attenuation member (A) has the shape of a wedge presenting a first beam entry face (A1) and a an opposed first beam exit face (A2), and the second energy attenuation member (B) has the shape of a wedge presenting a second beam entry face (B1) and an opposed second beam exit face (B2). The first and second beam entry faces are flat faces. The first and second beam exit faces are also flat faces. Furthermore, the dive unit (10) is configured for moving the first and second energy attenuation member in translation across the particle beam (50). Beyond the fact that they are easier to design and to manufacture, the advantage of wedge shaped attenuation members is that the amount of energy attenuation can be more freely and more accurately varied following to the second movement. In case the slopes of the two wedges are not the same, the first and the second speed may be different during the first movement in order to keep the total energy attenuation constant.

FIG. 7 schematically shows a 3D view of an even more preferred energy degrader according to the disclosure. It is identical to the one of FIG. 6, except that the first beam entry face (A1) is parallel to the second beam exit face (B2), the first beam exit face (A2) is parallel to the second beam entry face (B1), and that—during the first movement—the horizontal (X) component of the instantaneous first speed (VA1) is equal to the horizontal (X) component of the instantaneous second speed (VB1). This can for example be obtained easily by using a drive unit (10) as shown in FIG. 3 and by blocking the first motor (M1) at standstill during the first movement, so that the first energy attenuation member (A) does not move with respect to the second energy attenuation member (B) during the first movement.

In such a configuration, it will be easily understood that a total distance traveled by the particle beam (50) through the first and the second energy attenuation members—and therefore a total energy attenuation of the particle beam (50) by the energy degrader—remains constant during the first movement, and that the total energy attenuation of the particle beam (50) quickly varies following to the second movement.

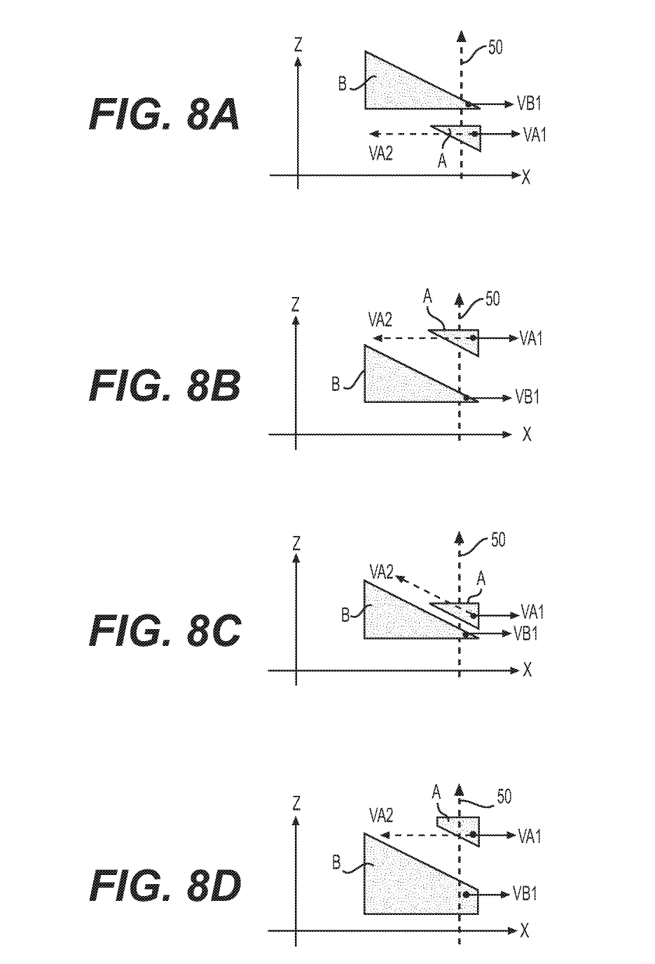

FIGS. 8a, 8b, 8c and 8d schematically show cross sections according to a plane parallel to the XZ plane of the energy degrader of FIG. 7 and according to various arrangements of the first and second energy attenuation member and of their respective movements. For the sake of clarity, the drive unit (10), although present, is not shown.

FIGS. 8b and 8c are more preferred arrangements since the beam enters and exits the energy degrader perpendicularly to its entry and exit surfaces.

FIG. 8c is an even more preferred arrangement since it allows keeping a smaller gap between the two wedges than in the case of FIG. 8b.

FIG. 8d illustrates the case where the wedges are truncated at one of their ends. As a general remark, it is to be noted that the lateral faces (the faces facing the YZ plane) of the wedges are preferably flat and parallel to each other, although this is not mandatory.

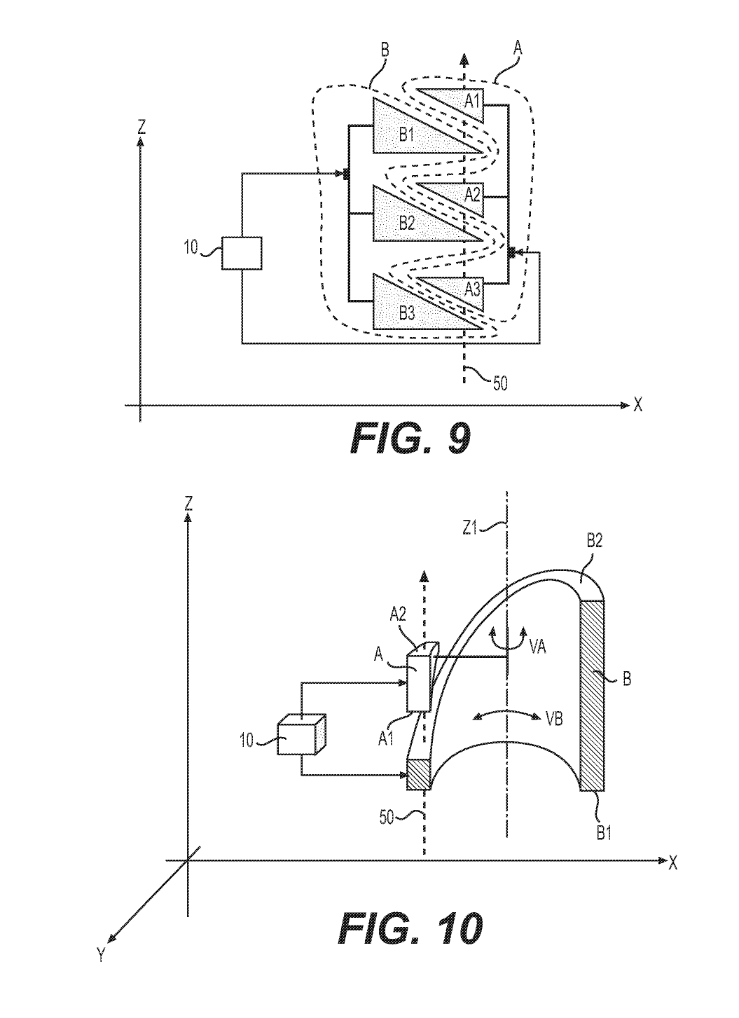

FIG. 9 schematically shows a variant of an energy degrader according to the disclosure. It is identical to the energy degraders described hereinabove, except that the first energy attenuation member (A) is subdivided into a plurality of mechanically interconnected first sub-members (Ai), and that the second energy attenuation member (B) is subdivided into a plurality of mechanically interconnected second sub-members (Bi). In this example wedge-shaped sub-members are shown, but any other shape may used as well.

When they have the shape of a wedge with mutually parallel beam entry and exit faces as shown in FIGS. 7 to 8, the energy attenuation members may for example have the following characteristics, particularly in the framework of a particle therapy system wherein the beam energy may be attenuated, from for example 230 MeV at the input of the degrader, to an energy ranging between 50 MeV and 230 MeV at the output of the degrader:

| | Second attenuation | First attenuation |
|---|---|---|
| | member (B) | member (A) |
| Width in X direction [cm] | 64 | 8 |
| Height in Z direction [cm] | 16 | 2 |
| Angle of wedge [°] | 14.04 | 14.04 |
| Depth in Y direction [cm] | 2 | 2 |
| Volume [cm3] | 1024 | 16 |
| Density [N/A] | 1.85 | 1.85 |
| Mass [g] | 1894.4 | 29.6 |

The first and second energy attenuation members as described hereinabove may also be fold around a central axis (Z1) which is preferably parallel the particle beam (50) at the location along the beam path where the particle beam (50) crosses the energy degrader. In such a case, the drive unit (10) is configured for driving the first (A) and second (B) energy attenuation members into rotation around the central axis (Z1) and according to the respectively described speeds (VA1, VB1, VA2), which are of course rotational speeds in this case.

FIG. 10 schematically shows an exemplary and preferred energy degrader which may be obtained this way as well as its position and orientation with respect to the particle beam (50). In this example, the first energy attenuation member (A) presents a first beam entry face (A1) having the shape of a portion of a first helicoidal ramp and a an opposed first beam exit face (A2) having the shape of a portion of a flat ring. The second energy attenuation member (B) presents a second beam entry face (B1) having the shape of a portion of a flat ring and an opposed second beam exit face (B2) having the shape of a second helicoidal ramp. The first helicoidal ramp is coaxial with the second helicoidal ramp. Preferably, the first helicoidal ramp is matching with the second helicoidal ramp, the first beam exit face (A2) is parallel to the second beam entry face (B1), and—during the first movement—the instantaneous first rotational speed (VA1) is equal to the instantaneous second rotational speed (VB1). This preferred configuration is in fact a rotational equivalent of the configuration of FIG. 7.

In any of the configurations described hereinabove, the drive unit (10) and the first and second attenuation members are preferably configured in such a way that a maximum gap between the first and second attenuation members in the course of the first and the second movements is smaller than 5 cm, preferably smaller than 1 cm, preferably smaller than 100 mm, preferably smaller than 10 mm, preferably smaller than 1 mm.

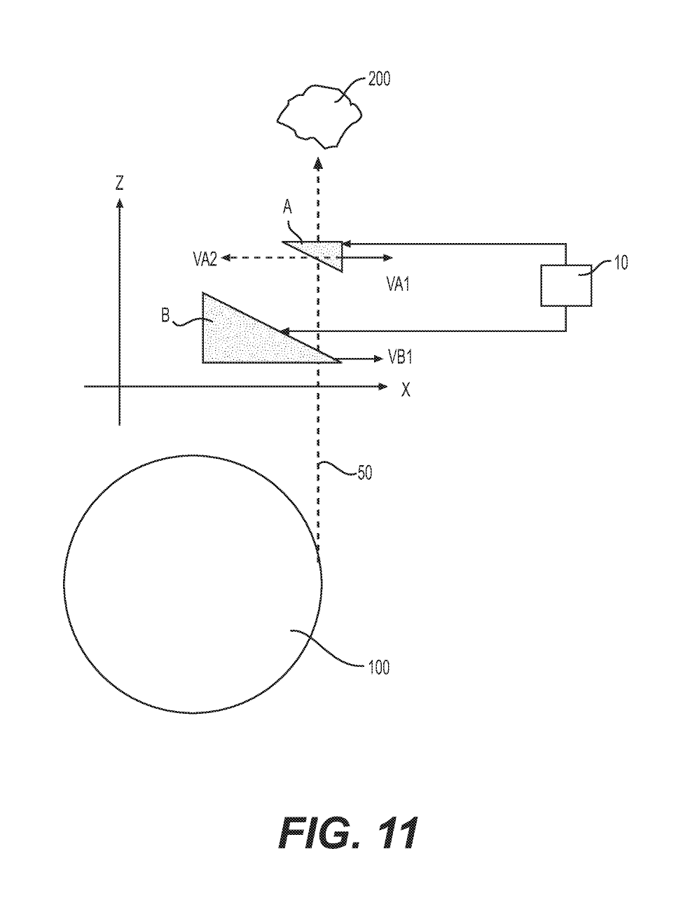

As schematically shown on FIG. 11, the disclosure also concerns a particle therapy system configured for irradiating a target (200) with a charged particle beam (50). Said particle therapy system comprises a particle accelerator (100) configured for outputting a charged particle beam (50), such as a beam of protons or carbon ions for example, and an energy degrader as described hereinabove for attenuating the energy of said charged particle beam (50) before it reaches the target (200). In the example of FIG. 11, the first and second energy attenuation members of the energy degrader are wedge-shaped, but any other shape as described hereinabove may be used as well. Preferably, the particle accelerator (100) is a fixed-energy accelerator. More preferably, the particle accelerator (100) is a cyclotron, for example a synchrocyclotron.

The present disclosure has described embodiments in terms of specific embodiments, which are merely illustrative and not to be construed as limiting. More generally, it will be appreciated by persons skilled in the art that the disclosed embodiments are not limited by what has been particularly shown and/or described hereinabove.

Reference numerals in the claims do not limit their protective scope.

Use of the verbs “to comprise”, “to include”, “to be composed of”, or any other variant, as well as their respective conjugations, does not exclude the presence of elements other than those stated.

Use of the article “a”, “an” or “the” preceding an element does not exclude the presence of a plurality of such elements.

Disclosed embodiments may also be described as follows: an energy degrader for attenuating the energy of a charged particle beam (50) and comprising two energy attenuation members (A, B) having different masses. The energy degrader further comprises a drive unit (10) configured to move simultaneously the two energy attenuation members at respectively a first and a second speed across the particle beam (50) during a first movement and to move the lightest of the two energy attenuation members at a third speed across the particle beam (50) during a second movement, the third speed being higher than the first speed.

1-15. (canceled)

16. An energy degrader for attenuating the energy of a charged particle beam, said energy degrader comprising:
a first energy attenuation member adapted to attenuate the energy of charged particles crossing said first attenuation member,
a second energy attenuation member adapted to attenuate the energy of charged particles crossing said second attenuation member,
wherein the first energy attenuation member has a first mass is smaller than a second mass of the second energy attenuation member,
a drive unit operably connected to the first and to the second energy attenuation member and configured for moving the first and/or the second energy attenuation member across the charged particle beam,
wherein the drive unit is configured to:
move simultaneously the first energy attenuation member at a first speed and the second energy attenuation member at a second speed across the charged particle beam during a first movement, and
move the first energy attenuation member at a third speed across the charged particle beam during a second movement,
wherein an average of the third speed over the second movement is larger than an average of the second speed over the first movement.

17. An energy degrader according to claim 16, wherein the mass first of the first energy attenuation member is less than half the second mass of the second energy attenuation member.

18. An energy degrader according to claim 16, wherein the average of the third speed over the second movement is more than double the average of the second speed over the first movement.

19. An energy degrader according to claim 16, wherein, during the first movement, a perpendicular component of the first speed is equal to a perpendicular component of the second speed.

20. An energy degrader according to claim 16, wherein the drive unit comprises:
a first motor configured for moving the first energy attenuation member at the first speed during the first movement and at the third speed during the second movement, and
a second motor configured for moving the second energy attenuation member at the second speed during the first movement.

21. An energy degrader according to claim 20, wherein the first motor includes a stator that is rigidly connected to the second energy attenuation member.

22. An energy degrader according to claim 16, wherein:
the first energy attenuation member has a wedge shape presenting a first beam entry face and an opposed first beam exit face;
the second energy attenuation member has a wedge shape presenting a second beam entry face and an opposed second beam exit face;
the first and second beam entry faces are flat faces;
the first and second beam exit faces are flat faces,
the first and the second movements are translational movements across the particle beam.

23. An energy degrader according to claim 22, wherein
the first beam entry face is parallel to the second beam exit face,
the first beam exit face is parallel to the second beam entry face, and
during the first movement, the first speed is equal to the instantaneous second speed.

24. An energy degrader according to claim 16, wherein:
the first energy attenuation member presents a first beam entry face having a shape of a portion of a first helicoidal ramp and a an opposed first beam exit face having a shape of a portion of a flat ring,
the second energy attenuation member presents a second beam entry face having a shape of a portion of a flat ring and a an opposed second beam exit face having a shape of a second helicoidal ramp,
the first helicoidal ramp is coaxial with the second helicoidal ramp, and
the first and the second movements are rotational movements around a central axis.

25. An energy degrader according to claim 24, wherein:
the first helicoidal ramp matches the second helicoidal ramp,
the first beam exit face is parallel to the second beam entry face, and
during the first movement, the first rotational speed is equal to the second rotational speed.

26. An energy degrader according to claim 16, wherein the drive unit and the first and second attenuation members are configured to form a maximum gap between the first and second attenuation members in the course of the first and the second movements that is smaller than 1 centimeter.

27. A particle therapy system comprising:
a particle accelerator configured to produce a charged particle beam, and
an energy degrader for attenuating the energy of the charged particle beam, said energy degrader comprising:
a first energy attenuation member adapted to attenuate the energy of charged particles crossing said first attenuation member,
a second energy attenuation member adapted to attenuate the energy of charged particles crossing said second attenuation member,
wherein the first energy attenuation member has a first mass is smaller than a second mass of the second energy attenuation member,
a drive operably connected to the first and to the second energy attenuation member and configured for moving the first and/or the second energy attenuation member across the charged particle beam,
wherein the drive unit is configured to:
move simultaneously the first energy attenuation member at a first speed and the second energy attenuation member at a second speed across the charged particle beam during a first movement, and
move the first energy attenuation member at a third speed across the charged particle beam during a second movement,
wherein an average of the third speed over the second movement is larger than an average of the second speed over the first movement.

28. A particle therapy system according to claim 27, wherein the particle accelerator is a fixed-energy accelerator.

29. A particle therapy system according to claim 27, wherein the particle accelerator is a cyclotron.

30. A particle therapy system according to claim 29, wherein the particle accelerator is a synchrocyclotron.
